(19) United States
(12) Patent Application Publication
(10) Pub. No.: US 2005/0169901 A1
(43) Pub. Date: Aug. 4, 2005

(54) Composition and methods afor diagnosis and treatment of cardiovascular disorders

(76) Inventors: Gerald Pang, New South Wales (AU); Patricia Lynne Conway, La Perouse New South Wales (AU); Robert Llewellyn Clancy, New South Wales (AU)

Correspondence Address: FISH & RICHARDSON PC, 225 FRANKLIN ST, BOSTON, MA, 02110, US

(21) Appl. No.: 10/512,356

(22) PCT Filed: Apr. 24, 2003
(86) PCT No.: PCT/AU03/00498

(51) Int. Cl. A61K 39/395; A61K 45/00

(52) U.S. Cl. 424/93.45; 424/93.5; 424/145.1

(57) ABSTRACT

The present invention relates to methods for diagnosis, prevention and treatment of cardiovascular disorders or associated disorders and, in particular, to the use in such methods of regulating cytokine levels or activity. Compositions and kits suitable for use in the methods are described.

### TECHNICAL FIELD

The present invention relates to methods for diagnosis, prevention and treatment of cardiovascular disorders or associated disorders and, in particular, to the use in such methods of agents capable of regulating cytokine levels or activity. Compositions and kits suitable for use in the methods are described.

### BACKGROUND OF THE INVENTION

Atheroma is the inflammatory process involving arteries that underpins coronary artery disease in particular and degenerative vascular disease in general. Data exists to support the concept that T lymphocytes drive inflammation within the atherosclerotic plaque. In particular, it has been reported that 2-10% of mononuclear cells in the plaque are T cells, two thirds of which are CD4+ve, and most of which express CD45RO, MHC class II, and IL-2R (Lamon et el Immunology Today 18 (1997) 272-7). Pro-inflammatory cytokines such as IL-1, IL-6, TNF-αand INF-γ are secreted from cells within plaque, as are cell modifying factors such as PDGF, MCP-1, and M-CSF, and proteolytic enzymes such as matrix metalloproteinase, e.g. collagenase and gelatinase B (Lamon et al, 1997).

The critical but complex relationship between T lymphocytes and macrophages within the plaque may be mediated in part by a receptor ligand couple through ligation of CD40L on activated plaque T cells by CD40 on macrophages (and other cells) to influence a range of outcomes including plaque remodelling, plaque rupture and antigen presentation (Lamon et al., 1997).

Recently, particularly microbes have been linked to the promotion of atheroma. The most characterised has been Chlamydia pneumoniae, through recent reviews have suggested that persistent in general may be linked to intimal inflammation and atheroma plaque growth (Saiku et al, Lancet 116 (1998) 983-5; Shar et al S Sfr Med J 82(1992) 158-61; Mejer et al JAMA 281(1991) 427). No data exists to clarify the basic mechanisms responsible for atheroma progression or processes whereby ‘epidemiologically-linked’ microbes facilitate atheroma growth.

There is a need for improved methods for assisting in the diagnosis of cardiovascular disorders and associated disorders which have basis in the immune response, e.g. coronary artery disease, and for compositions and methods for the prophylaxis and/or therapy of such conditions.

### SUMMARY OF THE INVENTION

It is an aim of the present invention to overcome or ameliorate one or more of the problems of the prior art, or to at least provide a useful alternative.

The present invention is based on the observation that certain “traditional” bacteria (probiotics) can be used for prophylaxis or treatment of cardiovascular disorders, such as atheroma. The observed positive effects of probiotics may be due to a shift in the cytokine profile of the subject e.g. from what is commonly known as a Th1 profile to a Th2 cytokine bias. For example, the concept that modern living atheroma is driven by altered cytokine patterns secondary to gut flora shifts, is consistent with the view that the essential difference between atheroma is developed versus developing countries is the excess amount of inflammation in plaque in developed countries.

It is thought that many factors modify the atheroma-promoting effect of inflammatory responses, in particular Th2 inflammatory responses (e.g. lipid levels, smoking, hypertension, etc.). Not wishing to be bound by any particular mechanism of action, we propose that the cause may be an environment effect on gut bacteria, replacing microbes that promote cytokine profile (e.g. the Th1 profile) such as Lactobacilli with other microbes that are linked to a different cytokine pattern (e.g. the Th2 response).

The new observation described in the present application provide a unique opportunity for diagnosis, prophylactics and therapies to detect and modify cardiovascular disorders, in particular in atheroma-prone or high load atheroma subjects. Diagnostics, prophylactics and therapy geared at additional microbes that may further exacerbate the cytokine imbalance one established (eg C. pneumoniae and H. pylori), are also specifically contemplated herein.

Thus, in broad terms the present invention is concerned with methods for diagnosing, detecting, preventing and treating cardiovascular disorders or disorders associated with cardiovascular disorders (in particular atheroma and coronary artery disease). Diagnostics can be based on the assessment of various markers and indicators of a specific cytokine response or profile (eg. a Th2 response) in blood (which interchanges with tissue spaces in the arterial wall). Probiotics suitable for use as therapeutic or prophylactic agents in the context of the present invention may affect the cytokine profile, for example, by promoting an increase or decrease in the level of a particular cytokine or cytokines. The cytokine may be cytokine involved in promoting what is commonly referred to as the Th1 response and/or suppressing a Th2 response.

According, in a first aspect, the present invention provides a method of prophylactic or therapeutic treatment of a cardiovascular disorder or an associated disorder comprising administering to a subject in need thereof an effective amount of an agent capable of regulating a cytokine associated with the disorder in the subject.

The cytokine may be any cytokine associated with a cardiovascular disorder or associated disorder. Preferred cytokines include interferon-γ(INF-γ), interleukin-4 (IL-4), interleukin-10 (IL-10), interleukin-12 (IL-12), interleukin-13 (IL-13) and TGF-β.

Regulation of the cytokine may be achieved by increasing or decreasing the level of the cytokine and/or by potentiating or inhibiting the activity of the cytokine. This form of regulation may, for example, be part of a more general switch from a Th2 to a Th1 cytokine profile. Regulation may be achieved by, for example, upregulation of a characteristic of a Th1 T-cell response relative to a cytokine profile of a Th2 T-cell response associated with the disorder. The upregulation of the cytokine profile characteristic of a Th1 T-cell response may be achieved by, upregulating Th1 T-cell response and/or suppressing Th2 T-cell response in the subject. Alternatively, the upregulating may be achieved by potentiating the activity of cytokines characteristic of a Th1 T-cell response and/or suppressing the activity of cytokines characteristic of a Th2 response. For example, for a Th1 response the characteristic cytokine may be interferon-γor interleukin-12, while for a Th2 response the characteristic cytokine may be interleukin-4, interleukin-10, TGF-βand/or interleukin-13.

A single agent or a plurality of agents may be administered to the subject to achieve the desired outcome.

Suitable agents for use in the invention may be, for example, microorganisms, or components, extracts or secreted products thereof capable of achieving the desired outcome. The microorganisms may, for instance, be yeasts, bacteria, and mixtures of these. Preferably, the microorganisms will be probiotic bacteria. Suitable probiotic bacteria may be selected from Lactobacillus spp, Mycobacterium spp and Bifidobacterium although other bacteria are also contemplated and the skilled addressee will be capable of identifying bacteria useful in the present invention. Lactobacilli having the capability of lowering cholesterol levels and/or suppressing the Th2 response are preferred. Particularly preferred are Lactobacillus acidophilus and Mycobacterium vaccae.

It will be understood that the microorganisms may be administered alive, inactivated or killed. Preferably, the bacteria are administered as viable organisms.

It will be understood that other agents capable of regulating a cytokine associated with a cardiovascular disorder or associated disorder may also be useful. In particular, useful agents may include, for example, antibiotics and binding fragments thereof. Anti-CD40 antibodies or binding fragments thereof are particularly preferred. In addition, other ligands for CD40 may be used.

The treatment outlined above can be combined with the administration of one or more pharmaceutically active agents used to treat underlying conditions that may exacerbate the cardiovascular disorder such as, for example, the lipid-lowering drugs, anti-hypertensive agents and anti-diabetic agents. The agent used to regulate the cytokine level or activity can be administered prior to, simultaneously with or subsequent to one or more such pharmaceutically active agents.

The methods of the invention may also be effective in subjects in which the disturbance in cytokine balance or the lack of an appropriate T cell response is exacerbated by bacterial infection, bacterial antigens, polyclonal activators (e.g. endotoxin etc.), super antigens (e.g. from colonising bacteria) or autoantigens (within the plaque of blood vessel walls). Particularly relevant to the present invention is infection by, or bacterial antigen from, Chlamydia pneumoniae, Helicobacter pylori or non-typable Haemophilus influenzae.

In a further aspect of the present invention there is provided a method of diagnosing, or evaluating susceptibility of a subject to, a cardiovascular disorder or associated disorder comprising detecting the level and/or activity of a cytokine associated with the disorder in the subject.

Preferably, the level and/or activity of the cytokine will be detected by analysis of circulating T-cells eg. measurement of any marker cytokine or cytokines characteristic of a particular T-cell response, such as interferon-γor IL-12 for a Th1 response or interleukin-4 and/or interleukin-13 for a Th2 response. Typically, an increase in the level or activity of a cytokine characteristic of a Th2 response and/or suppression in the level or activity of a cytokine characteristic of a Th1 response is indicative of the presence of, or susceptibility to, the disorder.

In a further aspect of the present invention there is provided a method of diagnosing a cardiovascular disorder or associated disorder or evaluating whether a subject is susceptible to the disorder, comprising:
(a) measuring one or more immunoglobulin levels affected by the disorder to obtain test data; and
(b) comparing the test data with reference data to evaluate whether the subject is susceptible to, or has, the cardiovascular disorder or associated disorder.

Preferably, the immunoglobulin is IgG and more preferably, the IgG2 subclass.

Preferably, the immunoglobulin is an antibody of the IgG2 subclass which is specific for pathogenic bacteria such as for example Chlamydia pneumoniae, Helicobacter pylori or non-typable Haemophilus influenzae. It will be clear to those skilled in the art that other specific antibodies may also be employed.

Preferably, a ratio of total IgG2 to IgG2 subclass specific antibody, or an altered ratio of total IgG2 subclass immunoglobulin to IgG2 subclass specific antibody will be used as an indicator of the presence of or susceptibility to the cardiovascular disorder or associated disorder.

The term ‘cardiovascular disorder’ is to be taken to encompass atheroma and degenerative vascular disease, and any cardiovascular condition or disease associated with inflammation of the coronary arteries including 1 to 3 coronary artery disease. Cardiovascular associated disorders include, but are not limited to, hypertension and increased cholesterol levels. Typically, the cardiovascular disorder will be a degenerative vascular disease and, in a preferred embodiment, atheroma.

Specifically, methods of the invention have application for the treatment of subjects suffering from atheroma (as determined by angiography) with minimal or extensive coronary atherosclerosis but stable clinical disease, as well as atheroma subjects with unstable clinical disease associated with recent myocardial infarction or unstable angina.

Compositions for use in the methods described herein are also specifically encompassed within the scope of the invention. Further, the use of the agents as described herein in the manufacture of a medicament or therapeutic composition for administering to a subject for the prophylaxis or therapeutic treatment of a cardiovascular disorder, is also specifically encompassed.

In addition, there are also provided kits for use in the methods of diagnosis or evaluation of the invention. A kit may, for instance, comprise one or more reagents for performing the assays such as antibodies, buffers, controls and instructions for use.

The feature and advantages of the present invention will be now be described hereinafter with reference to a number of preferred, non-limiting embodiments of the invention.

### BRIEF DESCRIPTION OF THE ACCOMPANYING DRAWINGS

FIG. 1 illustrates suppression of IL-4 secretion in whole blood by L. fermentum;

FIGS. 2A and 2B illustrate suppression of IL-4 secretion and potentiation of IFN-γ secretion by L. acidophilus, respectively;

FIGS. 3A and 3C illustrate secretion of IL-4 in C. pneumoniae seronegative and seropositive subjects with coronary vessel disease compared to normal subjects respectively;

FIGS. 4A and 4D illustrate secretion of IL-4 and IFN-γ in subjects with coronary vessel disease compared to normal subject respectively;

FIG. 5 illustrates the effect of Lactobacillus fermentum on atherosclerosis in mice fed in high cholesterol diet; and

FIG. 6 illustrates inhibition of IL-4 production by treatment of whole blood cultures with anti-CD40 monoclonal antibody.

FIG. 7. Secreted IL-4 levels in C57/B16 mice fed a high cholesterol diet or normal rat diet and dosed every second day with PBS, L. acidophilus strain VRI 001 (log 9 per dose) or L. acidophilus VRI 001 (0.5×10⁹)+L. fermentum VRI 002 (0.5×10⁹) per dose for 11 weeks.

FIG. 8. Secreted interferon-gamma in C57/B16 mice fed a high cholesterol diet or normal rat diet and dosed every second day with PBS, L. acidophilus strain VRI 001 (log 9 per dose) or L. acidophilus VRI 001 (0.5×10⁹)+L. fermentum VRI 002 (0.5×10⁹) per dose for 11 weeks.

FIG. 9. Fatty streak formation on the aortic arch of C57/B16 mice fed a high cholesterol diet and every second day with PBS, L. acidophilus strain VRI 001 (log 9 per dose) or L. acidophilus VRI 001 (0.5×10⁹)+L. fermentum VRI 002 (0.5×10⁹) for 11 weeks.

FIG. 10. Secretion of IL-4 levels in C57/B16 mice fed a high cholesterol diet for 12 weeks and dosed at week eight for the next 4 weeks every second day with PBS, L. acidophilus strain VRI 001 (log 9 per dose) or L. acidophilus VRI 001 (0.5×10⁹)+L. fermentum VRI 002 (0.5×10⁹).

FIG. 11. Levels of cholesterol in C57/B16 mice fed a high cholesterol diet for 12 weeks and dosed at week eight for the next 4 weeks every second day with PBS, L. acidophilus strain VRI 001 (log 9 per dose) or L. acidophilus VRI 001 (0.5×10⁹)+L. fermentum VRI 002 (0.5×10⁹).

FIG. 12. Fatty streak formation on the aortic arch of C57/B16 mice fed a high cholesterol diet for 12 weeks and dosed at week eight for the next 4 weeks every second day with PBS, L. acidophilus strain VRI 001 (log 9 per dose) or L. acidophilus VRI 001 (0.5×10⁹)+L. fermentum VRI 002 (0.5×10⁹).

### DETAILED DESCRIPTION OF PREFERRED EMBODIMENTS OF THE PRESENT INVENTION

It has been observed that the presence of significant atheroma results in elevated blood levels of IL-4 and a concomitant reduction in IFN-γ levels. This alteration in the cytokine balance may be indicative of a shift towards a Th2 response and is useful in the diagnosis of atheroma. The observation also provides a basis for treatments aimed at altering the T cell response (eg. towards a Th1 response) and thus, are beneficial in preventing and/or treating coronary arty disease and other cardiovascular disorders including atheroma which have bases in a similar underlying mechanism.

An example of possible therapeutic preparations contemplated herein are those which include probiotic bacteria (such as lactobacilli) which can alter the cytokine balance (eg. drive the cytokine balance back towards a Th1 response) and thus reduce progression of, prevent onset of, or reverse the cadiovascular disorder or associated disorder. However, other agents and compositions such as, for example, bacterial adjuvants as described below that have the ability to shift the cytokine balance (eg, from a Th2 response to a Th1 profile) are also useful in therapies for the conditions described herein.

Any method of detecting a cytokine change (eg a Th2 bias) in circulating T cells, whether directly or indirectly such as by monitoring downstream effects of cytokine bias such as IgG subclass variation or IgG subclass specific antibody variation as would occur in the production of antibody to C. pneumoniae or H. pylori (but not limited to those pathogens), would be useful as an indication of coronary artery disease. For example, IgG2 is relatively low when the cytokine patterns shift towards Th2. The thus altered ratio (or low levels) of total IgG2 subclass immunoglobulin or IgG2 subclass antibody specific for instance to C. pneumoniae or H. pylori, would indicate ‘atheroma-promoting’ cytokine bias.

Indeed, levels of immunoglobulins such as IgG2 subclass antibody may be measured and compared to reference levels or ratios to allow an evaluation to be made as to whether a subject is susceptible to a cardiovascular disorder such as atheroma, or otherwise has the disease. Suitable reference levels or ratios will generally be based on corresponding measurements obtained from healthy individuals and will typically comprise mean values derived from a representative cohort of the population in accordance with conventional methodology.

Further, methods of preventing, treating or reversing atheroma contemplated by the present invention include any treatment that shifts or otherwise alters the cytokine balance (eg. towards a Th1 response), such as the administration of probiotic bacteria (especially Lactobacilli species). For instance, Lactobacillus acidophilus can downregulate IL-4 and upregulate INF-γsecretion from T cells within the spleen (i.e. circulating cells) and thus have application in the treatment of atheroma and other such cardiovascular disorders. Other treatments include the administration of factors that suppresses cytokine secretion typical of a Th2 response or inhibits action of these cytokines, and/or any treatment that promotes secretion or activity of Th1 cytokines such as INF-γ.

It will be also be clear to those skilled in the art that any treatment that specifically modifies the level or pattern of cytokine secretion from circulating T cells specifically reactive to antigens (eg C. pneumoniae or H. pylori) or non-specific activating factors (eg polyclonal activators, endotoxin or superantigens) can be employed and is contemplated herein.

Further, treatments combining probiotics or other agents capable of altering the cytokine balance (eg. towards a Th1 response) with any existing therapy aimed at ‘risk factors’ eg. lipid-lowering drugs, anti-hypertensive agents and the like may also be usefully employed. Many additional factors drive atheroma (eg blood lipids, diabetes, hypertension, smoking) and the combination of therapies which alter cytokine balance with those which treat the underlying condition are also contemplated herein.

Typically, a sample will be obtained from the subject for evaluating T-cell cytokine profile and/or the T-cell response. The sample may be a whole blood sample, a cellular component of whole blood, isolated cells, or, for instance, a tissue biopsy sample suitable for assaying.

The microorganisms may be selected from bacteria and yeast strains including saccharomyces spp. such as Saccharomyces cerevisae and Saccharomyces boulardii. Preferably, the bacteria will be a probiotic bacteria. Alternatively, components, sonicates, extracts or secreted products, or mixtures thereof of the microorganism(s) may be used. Extracts include, for example, cell wall fractions. Components of the microorganism(s) may comprise antigens for instance, antigenic peptides and the like obtained by enzymatic treatments well within the scope of the sled addressee.

Bacteria may, for example be selected from, but not limited to, Lactobacillus species, lactic acid bacteria, Mycobacterium species and Bifidobacterium species. Even more preferred is the use of Lactobacillus acidophilus (L. acidophilus), Lactobacillus fermentum (L. fermentum) or Mycobacterium vaccae (M. vaccae), or components, extracts, sonicates, secreted product or mixtures thereof that are capable of altering cytokine levels or activity (eg. by inducing a Th1 cellular response). Specially preferred is L. acidophilus, L. fermentum, or M. vaccae which may be used live or as an inactivated preparation, as long as they are capable of inducing the desired response.

Preferably, L. acidophilus and L. fermentum is used as a live preparation. Other bacteria may also be used (whether they have probiotic effect or not), for example the well known adjuvating bacteria such as for example L. casei, L. plantarum, L. rhamnosus, Bifidobacterium breve and the like.

The dosage of the microorganism or extracts and the like thereof administered to the subject may vary according to the nature and severity of the cardiovascular disorder, whether the agent is administered for prophylactic or therapeutic purposes and the type of organism involved. The treatment parameters as well as the required dosage can be readily determined by the person skilled in the art.

Preferably, a microorganism or microorganism-containing composition will be in tablet or capsule form. However, it will be clear to those skilled in the art that the microorganism may be provided in a liquid or other form of solid preparations. In particular, the microorganism may also be provided as a food source such as a yoghurt or other dairy product, or similar non-dairy products based for example on soy.

The microorganisms or the like will generally be administered orally at regular intervals, and typically daily for the duration of the treatment period which may extend for a period of up to several months or more. Preferably, the microorganisms will be administered in a dosage of log 3 to log 12 per day. The dosage of probiotic bacterium when administered as live whole bacterium may be in the range of from about 1×10⁸ to about 1×10¹² organisms.

However, other agents capable of upregulating a cytokine profile (eg. a profile characteristic of a Th1 T-cell response) in accordance with methods of the invention may also be utilised. The skilled addressee will be able to readily identify such other agents by routine trial and experimentation on the basis of the teachings provided herein. Such other agents may include, for instance antibodies and binding fragments thereof.

In this regard, the present inventors have found that levels of blood T-cell secreted IL-4 associated with atheroma correlates with the extent of the coronary artery disease. This impressive correlation fits well with observations by the present inventors that T-cell mediated inflammation is driven by ligation of CD40L on CD4+ T-cells by CD40 on a range of structural and circulating cells including platelets. In particular, platelets appear to be an important factor for the production of IL-4 as a result of ligation of CD40L expression on activated CD4+ T-cells by CD40 expressed on the platelets.

Accordingly, administration of an agent capable of inhibiting ligation of CD40L with CD40 such as an antibody, and particularly an anti-CD40 antibody or binding fragments thereof, may alter the cytokine profile (eg. the cytokine profile characteristic of a Th2 response) in the patient. By binding ents is meant fragments of an antibody which retain the binding capability of the antibody and include Fab and (Fab′)₂ fragments as may be obtained by papain or pepsin proteolytic cleavage, respectively. In addition, other ligands for CD40 as will be known the skilled addressee or peptide fragments thereof may be administered for achieving the desired effect (eg. upregulation of a Th1 T cell response relative to a Th2 T cell response). Appropriate such ligands and agents can be readily identified utilising the methodology as disclosed in the accompanying Examples. Such agents may be administered intravenously, intramuscularly, or subcutaneously, or by any other route deemed appropriate.

Such agents and other agents like microorganism extracts, sonicates and the like may be formulated into pharmaceutical compositions incorporating pharmaceutically acceptable carriers, diluents and/or excipients for administration to the intended subject. The dosage of such other active agents will typically be in accordance with conventional treatment regimens for their use taking into account such factors as age, weight, nature of the condition being treated and the general health of the subject as will be readily appreciated.

Pharmaceutical forms include aqueous solutions suitable for injection, and powders for the extemporaneous preparation of injectable solutions. Such injectable compositions will be fluid to the extent that syringability exists and typically, will be stable to allow for storage after manufacture. The carrier may be a solvent or dispersion medium containing one or more of ethanol, polyol (eg glycerol, propylene glycol, liquid polyethylene glycol and the like), vegetable oils, and suitable mixtures thereof. Fluidity may be maintained by the use of a coating such as lecithin, by the maintenance of the required particle size in the case of a dispersion and by the use of surfactants.

Injectable solutions will typically be prepared by incorporating the active agents in the desired amount in the appropriate solvent with various offer components enumerated above. Generally, dispersions will be prepared by incorporating the active agents into a vehicle which contains the dispersion medium and other components. In the case of powders for the preparation of injectable solutions, preferred methods of preparation are vacuum drying and freeze-drying techniques which yield a powder of the active agent.

For oral administration, agents may be formulated into any orally acceptable carrier deemed suitable. In particular, the active ingredient may be formulated with an inert diluent, an assimilable edible carrier or it may be enclosed in a hard or soft shell gelatin capsule. Alternatively, it may be incorporated directly into food as indicated above. Moreover, an active agent may be used in the form of ingestable tablets, troches, capsules, elixirs, suspensions, syrups, and the like.

A composition of the invention may also incorporate one or more suitable preservatives such as sorbic acid. In many cases, a composition may furthermore include isotonic agents such as sugars or sodium chloride.

Tablets, troches, pills, capsules and the like may also contain one or more of the following: a binder such as gum tragacanth, acacia, corn starch or gelatin; excipients such as dicalcium phosphate, a disintegrating agent such as corn starch, potato starch, alginic acid and the like; a lubricant such as magnesium sterate; a sweetening agent such as sucrose, lactose or saccharin or a flavoring agent. When the dosage unit form is a capsule, it may contain in addition to one or more of the above ingredients a liquid carrier. Various other ingredients may be present as coatings or to otherwise modify the physical form of the dosage unit. For instance, tablets, pills or capsules may be coated with shellac, sue or both. In addition, an active agent may be incorporated into any suitable sustained-release preparation or formulation.

Pharmaceutically acceptable carriers, diluents and/or excipients include any suitable conventionally known solvents, dispersion media and isotonic preparations or solutions. Use of such ingredients and media for pharmaceutically active substances is well known. Except insofar as any conventional media or agent is incompatible with the active agent use thereof in therapeutic and prophylactic compositions is contemplated. Supplementary active ingredients can also be incorporated into the compositions if desired.

As will be appreciated, the amount of agent or agents in such compositions will be such that a suitable effective dosage will be delivered to the subject taking into account the proposed mode of administration.

Dosage unit form as used herein is to be taken to mean physically discrete units suited as unitary dosages for the subject to be treated, each unit containing a predetermined quantity of active agent calculated to produce the desired therapeutic or prophylactic effect in association with the relevant carrier, diluent and/or excipient.

The agent may be administered in conjunction with one or more antibiotics or one or more other pharmaceutically active agents for treating the cardiovascular disorder or any underlying condition that exacerbates the disorder, and may be administered prior to, simultaneously with or subsequent to antibiotic therapy or therapy with other active agents.

### EXAMPLES

### Example 1

### Lactobacillus Inhibits IL-4 Secretion

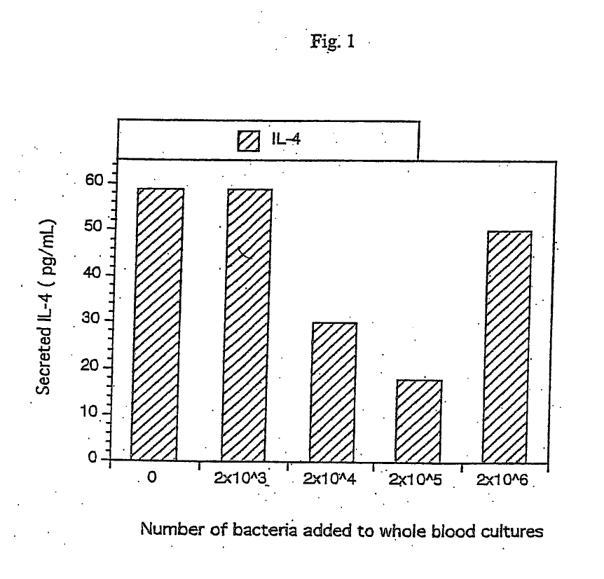

To determine whether Lactobacillus has the capacity to regulate IL-4 production, graded does of lactobacillus fermentum (strain VRI 002 available from the Culture Collection of the School of Microbiology and Immunology at the University of New South Wales, Sydney, Austria) were added to cultures containing equal volumes of heparinized whole blood from a normal healthy subject and AIM-V serum free medium. Control cultures contained medium alone. All cultures were stimulated with Con A (5 ug/ml). After incubation for 24 hrs, the amount of secreted IL-4 was determined by capture IL-4 ELISA. As shown in FIG. 1, IL-4 secretion was inhibited in a dose dependent manner in the presence of L. fermentum with maximal effect occurring at 2×10⁵ bacteria per culture. This data indicates that Lactobacillus fermentum is effective in down-regulating IL-4 mediated inflammation (eg. inflation associated with a Th2 response).

### Example 2

### Effect of Probiotic Bacteria on Cytokine Levels

To determine whether probiotic bacteria can regulate a cytokine response (eg. down-regulate a Th2 and up-regulate a Th1 cytokine response), C57/B16 mice were fed intragastrically, various numbers of Lactobacillus acidophilus (stain VRI 001 available from the Culture Collection of the School of Microbiology and Immunology, University of New South Wales, Sydney, Australia) using a feeding needle on consecutive days for 2 weeks, after which they were sensitised with 8 μg of ovalbumin (OVA) and aluminium hydroxide in 0.2 mL phosphate-buffered saline administered by peritoneal injection. The mice were further fed ten times with L. acidophilus every two days for two weeks before they were sacrificed. Lymphocytes were isolated by teasing spleens through a sieve, washed with PBS, and resuspended at 10×10⁶ cells/ml culture medium.

One mL aliquots of the cell suspension were then dispensed into wells of a 24-well flat-bottomed microtitre plate and stimulated with OVA (5 μg/mL). After incubation for 4 days the supernatants were collected and assayed for IL-4 and IFN-γproduction by standard ELISA techniques using IL-4 or IFN γmonoclonal antibody pairs.

Briefly, wells of a 24-well microtitre plate were coated with a capture anti-IL-4 antibody. After incubation at room temperature for 1 hr, the wells were washed and biotinylated anti-IL antibody was added to each well. Following incubation for a further 1 hr, the wells were washed and strepavidin-peroxidase conjugate was added to each well. After incubation for 30 mins, the wells were washed and then TMB substrate was added. The colour development was read at 450/620 nm in an ELISA plate reader. The level of IL-4 in unknown samples was quantitated by interpolation using a standard curve. A similar procedure was used for measurement of IFN-γ.

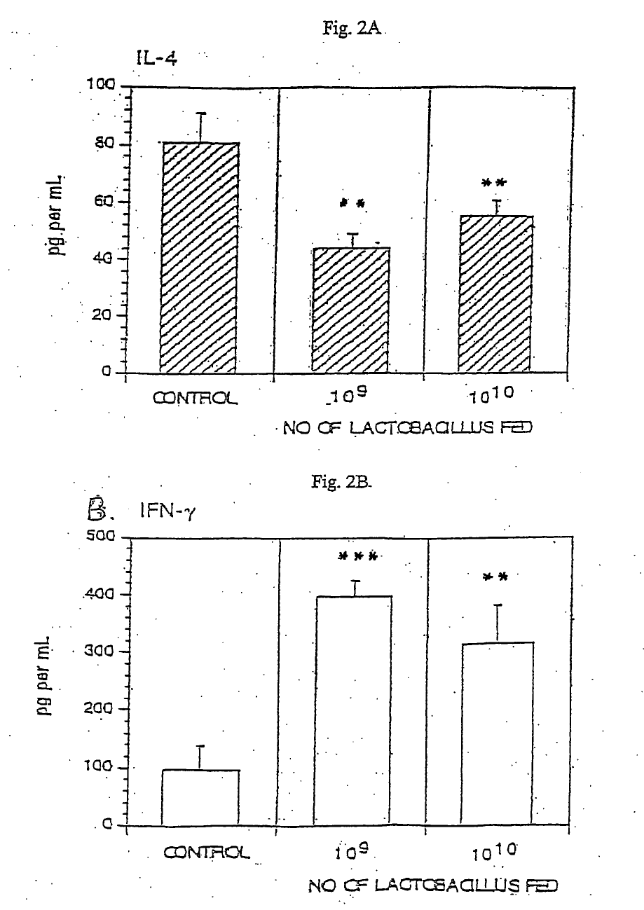

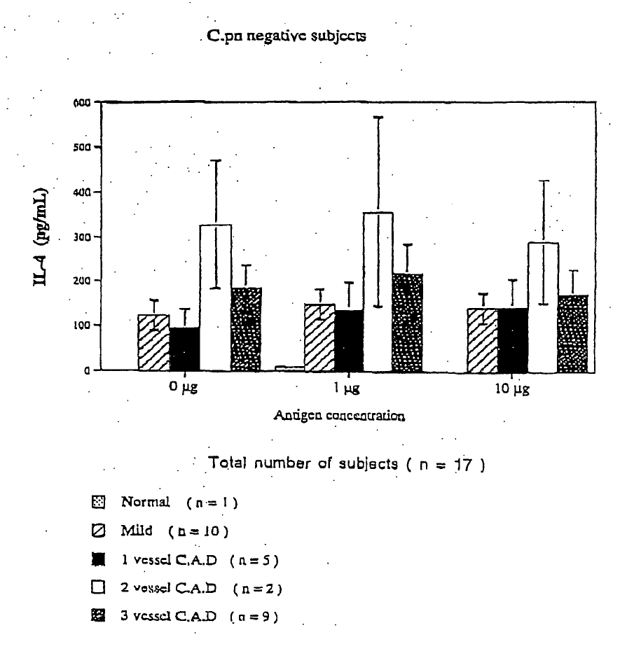

The results are shown in FIG. 2A and FIG. 2B. As can be seen, FIG. 2A demonstrates that feeding L. acidophilus resulted in the suppression of IL-4 production in a dose-dependent manner whereas FIG. 2B shows that production of IFN-γwas enhanced. Accordingly, increased production of secreted IL-4 in whole blood correlates with severity of disease in subjects with coronary artery disease.

### Example 3

### Subject Selection and Measurements

3.1 Subjects. Subjects presenting at the John Hunter Hospital (Newcastle, Australia) were selected following angiographic study. Risk factors were recorded (lipid profile, hypertension, diabetes, smoking, family history). The following groups were identified: (a) minimal coronary atherosclerosis (n=100); (b) extensive coronary atherosclerosis (>50% three major vessel involvement) with stable clinical disease (n=100), and (c) extensive coronary atherosclerosis−unstable clinical disease (n=100) (recent myocardial infarction or unstable angina).

Blood (20 ml) was taken following angiography from the selected subjects for antibody and T cell studies. The number of angiographic studies at the John Hunter Hospital (Newcastle, Australia) is about 30-40/week with the distribution being approximately 10-15% with normal arteries or nal disease and 20-30% with triple artery disease, of which about one third has unstable clinical disease and two thirds have stable clinical disease.

3.2 Anti-Chlamydia Pneumoniae Antibody. The antibody was detected by a micro-immunofluorescence best for immunoglobulin IgG to C.pn-specific antigen (Chlamydia-cel Pn kit, CeLLabs Pty Ltd, Australia). IgG subclass antibody was detected using specific IgG subclass antisera.

3.3 T-cell proliferation. Whole blood lymphocyte culture was performed in triplicate in 96-well round-bottomed microtitre plates. Heparinised blood was diluted 1:1 (v/v) with AIM-V serum free-medium containing graded amounts (0.1, 1.0, 10 μg/ml) of Chlamydia pn elemental bodies Q) prepared as described below. All subjects we stimulated in addition with C. trachomatis or EB antigen (0.1, 1.0, 101 μg/ml) as an ‘irrelevant’ antigen control. After five days at 37° C. in 5% CO₂, titrated thymidine (0.5 μCi per culture) was added for the final six hours before harvesting and counting.

3.4 Cytokine production. Cytokine-based whole blood assays for detection of EB-reactive T cells were used. Heparinised blood was diluted 1:1 (v/v) with AIM-V medium with or without various concentrations of EB antigens in wells of a 96-well round-bottomed microtitre plate. For measuring the production of IL-4, some wells were pre-coated with a capture monoclonal anti-IL4 antibody (Endogen, CSL). The cultures were incubated at 37° C. in a 5% CO ₂ atmosphere for 24-48 hours after which time the plasma supernatants were collected for IL-2, IL-10 and IFN-γ assays (Endogen kits, CSL). Captured IL-4 together with appropriate standards were directly determined in the wells following washing and the addition of developing anti-IL-4 antibody as described in the assay kit. The whole blood assay for measuring antigen-reactive T cells and cytokine production profiles had been validated for studies in human subjects with H. pylori infection.

3.5 Preparation of elemental bodies from Chlamydia pn. A. HeLa cell 229 adapted C.pn Kajaani strain obtained from Professor P Saikku (University of Helsinki, Finland) was grown in HeLa cells in culture flasks containing RPMI 1640 medium supplemented with 5% foetal calf serum (FCS) and streptomycin at 37° C. in a 5% CO₂ humidified atmosphere. Chlamydia elemental bodies were isolated from cultured cells after three days. The cells were detached from the flask using a sterile scraper, washed and suspended in phosphate buffered saline (PBS) and the inclusion bodies disrupted by sonication. After removal of cell debris by centrifugation, the EB material was collected by ultracentrifugation at 30,000 g. The EB material was then resuspended in PBS and layered onto a 30-60%. Nycodenz solution (Nycomed, Norway). After centrifugation, the EB materials collected above the 60% gradient were washed and then inactivated with 1% formaldehyde for 24 hours. After extensive washing, the BB material was resuspended in PBS and the protein concentration determined (Pierce Protein Kit). EB antigens obtained from Professor Saikku and colleagues were also used in the study for comparison. A similar method was used for an elemental body antigen preparation from C. trachomatis (with samples again being provided by Professor P Saikku).

3.6 Specific cloned proteins. Cloned antigens from C.pn supplied by Drs. Saikku and Makela (Finland-above) were tested for cytokine balance (above). The cloned antigens comprised MOMP, OMP2 and HSP60 as recombinant proteins produced in B. subtilis. These were tested at 1 μg/ml.

In particular, heparinised whole blood was collected from patients with coronary atherosclerosis who were either seropositive (n=17) or soronegative (n=27) for C pneumoniae. After incubation overnight at 37° C. as above, secreted IL-4 was measured by capture ELISA while IFN-γ was measured in plasma supernatant.

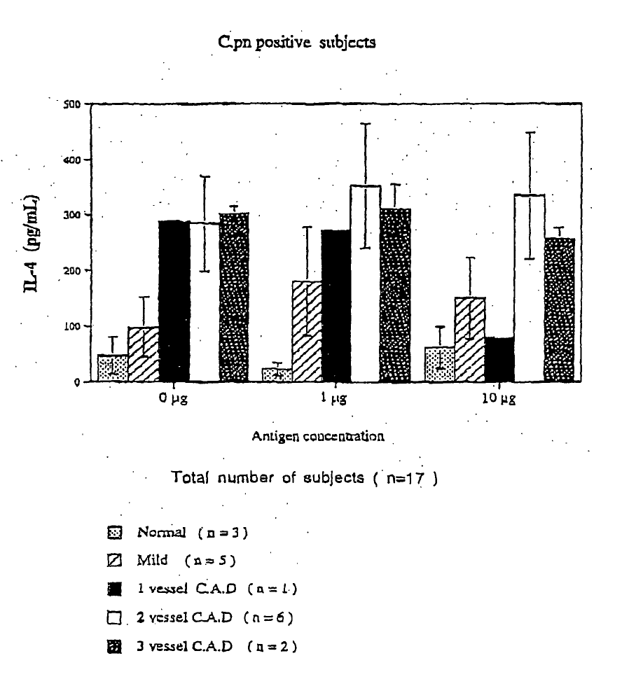

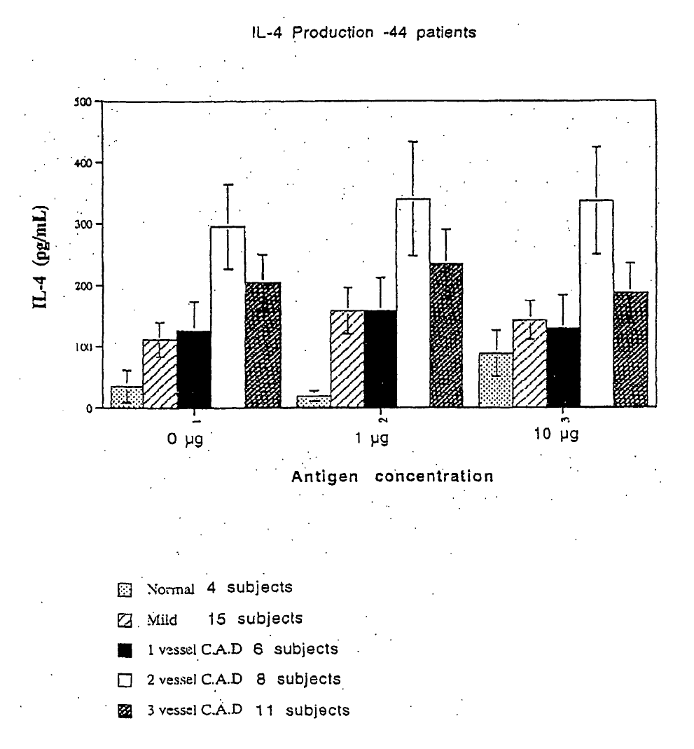

As shown in FIGS. 3A and 3B, higher levels of IL-4 were detected in subjects with 2-3 coronary vessel disease compared to subjects with mild or 1 vessel disease. Low to undetectable levels were observed in normal subjects. In C pneumoniae seropositive subjects, higher levels of secreted IL-4 were detected in those with 1-3 vessel disease compared to seronegative subjects especially those with 1 vessel disease, suggesting that increased production of secreted IL-4 is associated with infection status. However, in all subjects studied, IL-4 secretion was not dependent on stimulation with C pneumoniae antigens in culture, indicating that spontaneous production of IL-4 was a result of activated T-cells in vivo which are no longer responsive to further antigen stimulation in culture. When the data from the 44 subjects were combined the results were similar in that irrespective of antigen stimulation the levels of secreted IL4 in whole blood cultures correlated with the extent of disease.

### Example 4

### Pattern of Spontaneous T Lymphocyte Activation

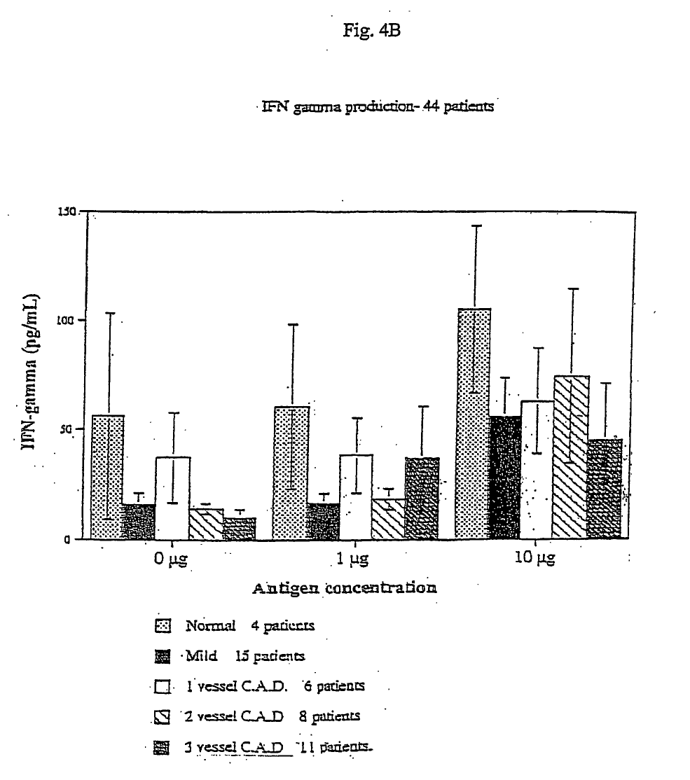

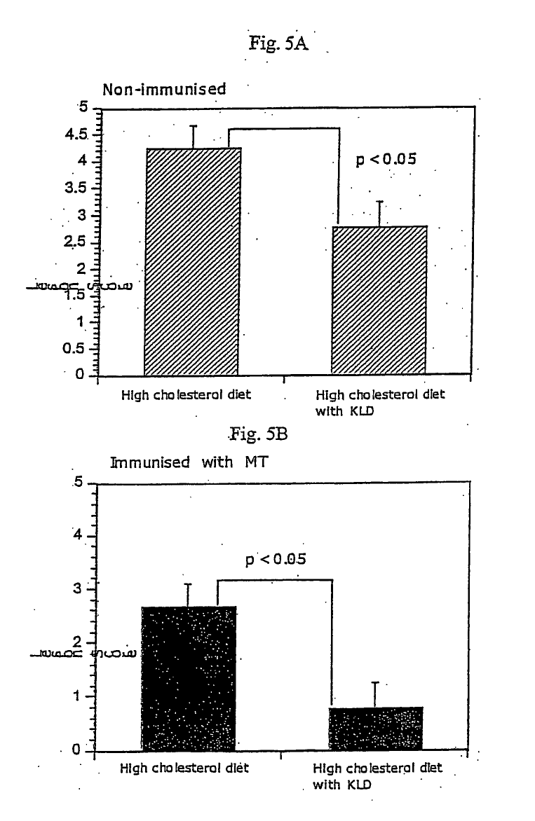

In marked contrast there was inverse relationship between secreted IL-4 and IFN-γ production (see FIGS. 4A and 4B). However, there was no correlation between levels of IFN-γ and the severity of disease indicating the inflammatory response in atheroma is driven by CD4+ Th2 helper cell-mediated inflammation with upregulation of IL-4.

In particular, the results of spontaneous cytokine production show a significant difference between those with ‘normal’ coronary angiograms and those with two or tree vessel disease (representing ‘high load’ atheroma), with those defined as mild or minimal coronary atherosclerosis being intermediate in amount of IL-4 produced. With respect to INF-γ, a difference between normal and ‘atheroma-detected’ subjects was found to be present, with the ‘normal’ subjects have higher levels. Differences between mild and severe atheroma for INF-γ is less marked than is the level of difference seen with IL-4. Taken together, these results clearly show that there is a shift in the cytokine balance correlating with the amount of atheroma.

It is concluded that subjects with a ‘set’ towards responding to stimuli of T cells with a certain cytokine response (eg. Th2 response), promote excessive accumulation of atheroma in blood vessel walls, as a result of the pathways of the inflammatory response linked to Th2 T cell activation. As cytokines measured here are spontaneously secreted from T cells in whole blood culture, activation has occurred in-vivo. Stimuli could include polyclonal activators (e.g. endotoxin from gut flora), super antigens (e.g. form colonising bacteria), autoantigens (including antigens within the plaque or blood vessel wall) or specific antigens, especially from microbes in a colonising or parasitic relationship with the host (e.g. Chlamydia pneumoniae, Helicobacter pylori, non-typable H. inflenzae etc). The latter is consistent with the view that “chronic infection unrelated to particular microbial species” is a ‘risk factor’ for atherosclerosis progression rather than C. pneumoniae having an unique antigenic role (Groyston J T, Kuo Coulson A S et al, Circulation (1995) 92: 3397- 3400; Bachmaier K, Neu N et al., Science (1999) 283: 1335-1339; Mejer D, Derby L E et al, JAMA (1999) 18: 272-277). In addition, the data in FIGS. 3A and 3B show a trend towards greater ‘Th2-polarisation’ in cultures stimulated with C. pneumoniae antigen, consistent with the notion that within the context of a Th2 set of the immune system, particular microbes may enhance the drive towards a Th2 response and thus further progress the atheroma plaque. Circulating cells would interchange with those included in atheroma plaque. Thus, chronic infection can exacerbate the na bias in subjects with significant atheroma. However, the present data on subjects with and without Chlamydial infection show that the basic “set” of Th2 cytokines is independent of Chlamydial infection (although the infection may exacerbate the bias as mentioned above).

This study supports the conclusion that the pattern of spontaneous T lymphocyte activation correlates with the amount of atheroma generally, but in particular in the coronary arteries.

### Example 5

### Effect of Feeding Lactobacillus on Atherosclerosis in Mice Fed a High Cholesterol Diet

The effect of a high cholesterol diet on the development of atherosclerosis as assessed by the formation of fatty streak in the aortic sinus (root) of mice was determined. The diet contained the following ingredients:

| | g/100 g |
|---|---|
| Sucrose | 51.3 |
| Casein (acid) | 20.0 |
| Canola oil | 1.00 |
| Cocoa butter | 15.00 |
| Cellulose | 5.10 |
| DL-methionine | 0.30 |
| AIN93G minerals | 3.50 |
| AIN93G Vitamins | 1.00 |
| Choline Chloride 50% w/w | 1.00 |
| Sodium Cholate | 0.50 |
| Cholesterol | 1.00 |
| DL α-Tocopherol acetate | 0.26 |

Briefly, C57/B16 male mice (8 weeks old) were placed on a high cholesterol diet (HCD) or a cholesterol free normal diet, and with free access to drinking water. Groups of mice (n=10) were fed HCD for one week and then placed on a feeding redden comprising Lactobacillus fermentum (VRI 002). The dose was administered oro-gastrically 3 times per week with a 200 μl sample of a washed bacterial suspension from an overnight culture resuspended to give a final density of between log 9.5 and log 10.5 organisms. Control mice were dosed with 200 μl of saline alone. After 5 weeks, two groups of nice were immunised subcutaneously with 0.1 mL of 5 mg/mL killed Mycobacterium tuberclosis (MT, Difco) emulsified in incomplete Freund's adjuvant. The rationale for th immunisation step was based on a recent report which suggests that activation of the immune system by immunisation with killed bacteria can lead to the acceleration of fatty streak formation in the aorta sinus (George J et al. Ateriosclerosis, Thrombosis and Vascular Biology, 1999, 19: 505-510).

All mice were sacrificed at 7 weeks after commencement of the HCD and probiotic treatment. Blood was collected by cardiac puncture. The heart was removed en bloc and the upper section containing the aortic sinus (root) was excised and fixed in 10% formalin in PBS. After fixing overnight in formalin/PBS, the tissue was embedded in OCT medium and frozen before sectioning in a cryostat. Six to seven sections (810 μm thick) were taken and stained with oil Red 0. Lesion areas per section were scored by a blind observer. A 0-5 lesion scoring system was adopted according to the presence of fatty streak formation. As show in FIG. 5A, mice fed HCD alone had more formation of fatty streak than those treated with Lactobacillus, Similar results were obtained with mice immunised with MT (see FIG. 5B) although in these mice the amount of lesion was lower than non-immunised groups, suggesting that immunisation may lit atherogenesis.

### Example 6

### IL-4 Production in Whole Blood Cultures from Patients with Coronary Artery Disease is Inhibited by Anti-CD40 Monoclonal Antibody

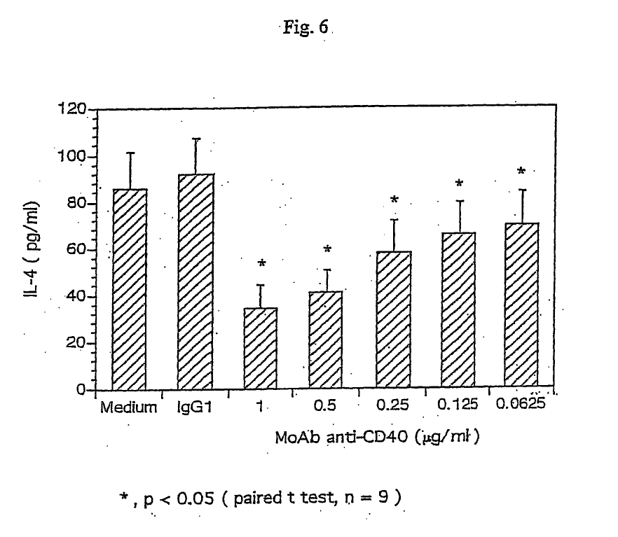

Heparinised blood was collected from subjects with coronary artery disease and cultured in equal volume of serum-free AIM-V medium (300 μL total volume) containing graded concentrations of anti-CD40L antibody in a 96-well flat-bottomed coated with anti-IL4 antibody. Control cultures contained medium alone or a mouse IgG1 isotype control. After incubation for 24 hrs, the amount of IL4 secreted was measured by a capture ELISA assay. As shown in FIG. 6, IL-4 production was inhibited by anti-CD40 in a dose-dependent manner compared with control (p<0.05 for 9 subjects) while the addition of mouse IgG1 isotype control or anti-CD40L (data not shown) had no effect. The result showed that the engagement of CD40 is critical for the production of IL4 whole blood culture.

### Example 7

7.1 Background

There is growing evidence from recent studies in humans and in experimental models of hypercholesterolemia that the immune system is specifically activated by atherosolerosis-associated circulating non-cellular and cellular elements to significantly modulate the initiation or progression of lesion development. For example, hypercholesterolemia can alter the switch from Th1 to Th2 immune response associated with lesion development (Zhou X et al J Clin Invest 1998; 101; 1717-25). Our studies have shown for the first time in humans that a Th12 response characterised by elevated IL4 secretion in circulating T cells is associated with more severe disease involving 2-4 vessels whereas those with normal to mild and a single vessel disease have undetectable to low IL-4. These findings were supported by recent studies in gene knockout mice susceptible to atherosolerosis showed that deficiency in IL-4 reduced atherosclerotic lesion formation (VL King et al Atheroscler Thrombosis Vasc Biol. 2002; 22: 456-461; J George et al., Circ. Res. 2000; 86: 1203-1210). However, it is unclear whether a high cholesterol diet is associated with increased secretion of IL4 in circulating activated T cells and lesion development in the artery of wild-type C57/B16 mice. The following is a summary of the findings:

7.2 Study Protocols

Aim 1

To determine whether a high cholesterol diet (HCD) leads to incased production of secreted IL-4 in circulating activated T cells and whether mice on HCD have lower secreted IL-4 when also fed probiotic bacteria.

Methods and Results

Eight weeks old CS57/B16 female mice were fed a high cholesterol diet for 11 weeks during which time they were fed by gavage with 0.2 ml of VRI101 (Lactobacillus acidophilus) or a combination of two probiotics (VRI 101 plus VRI102 Lactobacillus fermentum) at 1×10 ⁹/mL in PBS every 2 consecutive days. Mice fed a normal diet (rat chow) were fed PBS. One day after the final treatment, the mice were sacrificed by CO₂ overdose. Blood was collected in heparin tube by cardiac puncture and cultured with or without Con A (0.1 μg/ml) in AIM-V medium for 22 has at 37° C. in wells of a 96-well flat-bottomed microtitre plate. Secreted IL-4 was measured by an in situ capture ELISA assay. The formation of fatty streak formation in the aortic sinus was assessed by histology. Serial frozen sections were obtained and stained with O Red oil. The lesion size was measured by microscopy using computer-assisted morphometry and was expressed as area in μM². The data shown were from mean values for mice on normal diet (n=7), HCD (n=9), VRI101 (n=9) or combination (n=10). (NOTE: strain VRI101 is also known as VRI001 and the two strain names have been used interchangeably in this specification.)

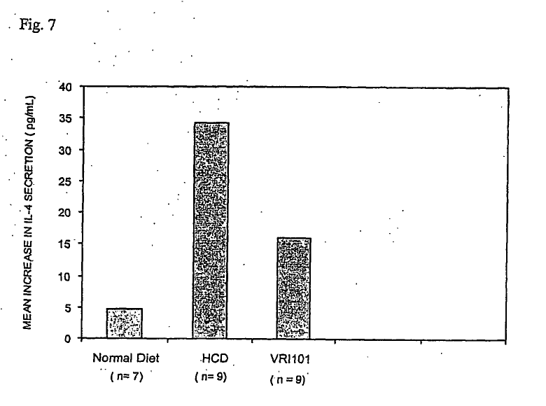

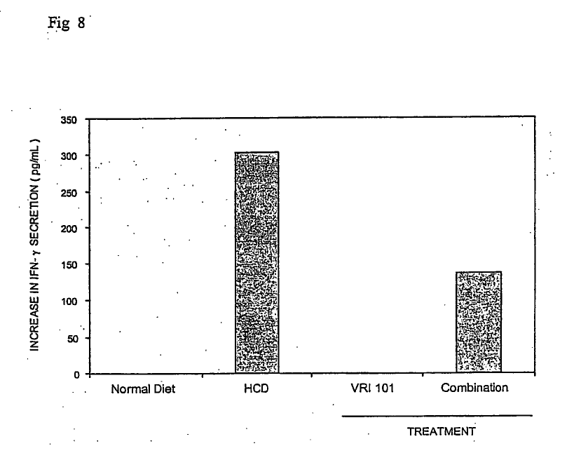

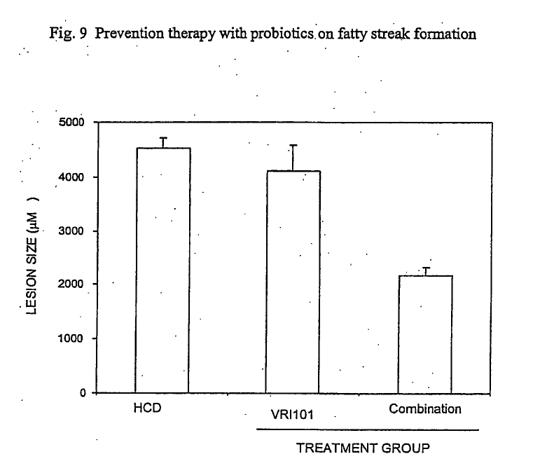

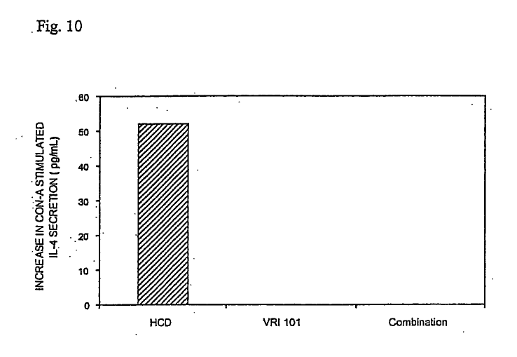

As shown in FIG. 7, mice fed HCD had elevated levels of secreted IL4 compared with mice fed normal rat chow, VRI101 alone had no significant effect on secreted IL-4 but the combination of probiotics reduced IL-4 secretion. A reduction in IFN-γ secretion was noted with VRI 101 or the combination (FIG. 8). Furthermore, reduction in secreted IL4 and IFN-γ correlated with reduction in fatty steak formation (FIG. 9). Taken together, the data show that a prevention therapy consisting of a combination of probiotics is effective in down regulating atherosclerotic-associated inflammatory cytokines and in reducing lesion formation.

7.4 Aim 2

To determine whether probiotics have an effect on cytokine production and lesion development in mice following a high cholesterol diet.

Methods and Results

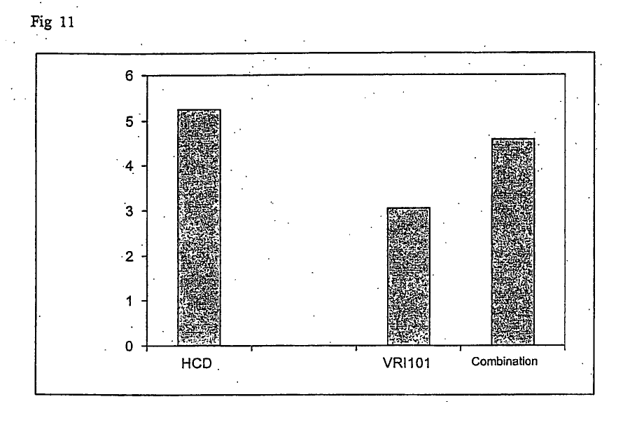

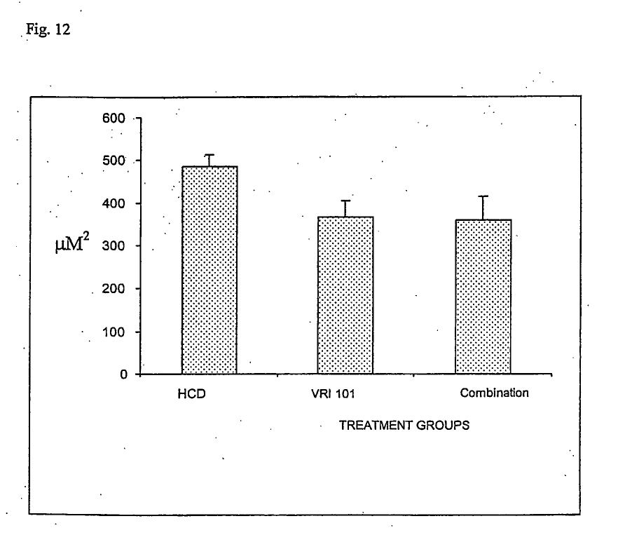

C57/B16 female mice (8 weeks old) were fed a high cholesterol diet for 8 weeks after which time they were treated on every 2 consecutive days with VRI101 or the combination of VRI101 and VRI102 for 3 weeks. Control HCD mice were fed PBS alone. One day after the last treatment, the mice were sacrificed. Heparinised blood was collected and stimulated in culture with or without Con A (0.1 ug/mL). Secreted IL-4 was measured by capture ELISA and IFN-γ in plasma supernatant was measured by ELISA. Fatty streak formation in the aortic root was assessed by histology. Frozen sections were stained with Oil Red O and haematoxylin. Fatty steak was measured by morphometry and expressed as area in μM ². The results shown were mean values for mice fed PBS (n=7), VRI101 (n=10) or the combination (n=10). The results in FIG. 10 showed that feeding hypercholesterolemic mice led to an abrogation of IL-4 secretion by Con-activated circulating T cells from mice fed VRI 101 or the combination. Reduction in secreted IL-14 also led to lower levels of plasma cholesterol in mice fed VRI 101 or the combination but not in untreated HOD-fed mice (FIG. 11). Similarly, a reduction in fatty streak formation was also noted in mice fed VRI 101 or the combination (FIG. 12).

### Example 8

### Reduction of Cholesterol In Vitro During Growth of Lactobacillus

Lactobacillus fermentum strain VRI 102 (PCC), deposited in the University of New South Wales Culture Collection was grown in MRS broth (Mann, Rogosa and Sharpe, Oxoid) in the presence of cholesterol and the percentage of cholesterol removed from the growth medium after overnight growth at 37° C. was determined. The growth of L. fermentum removed 43% cholesterol from the medium.

### Example 9

### Lowering of Blood Pressure in a Male with Consistent High Blood Pressure by Oral Administration of Lactobacillus

A male, 50-55 age group, in good health except for consistent high blood pressure was dosed with around log 10 cfu viable Lactobacillus fermentum per day for two months. Blood pressure prior to commencement of the treatment was 149/110 and after treatment it was 120/80.

Although the present invention has been described with reference to preferred embodiments, the skilled addressee will understand that numerous variations and modifications are possible without departing from the scope of the instant invention.

1. A method of prophylactic or therapeutic treatment of a cardiovascular disorder or an associated disorder comprising administering to a subject in need thereof an effective amount of an agent capable of regulating a cytokine associated with the disorder in the subject.

2. A method according to claim 1 wherein the cytokine is selected from the group consisting of: interferon-γ (INF-γ), interleukin-4 (IL-4), interleukin-10 (IL-10) interleukin-12 (IL-12), interleukin-13 (IL-13) and TGF-β.

3. A method according to claim 1 wherein regulation of the cytokine is achieved by increasing or decreasing the level of the cytokine and/or by potentiating or inhibiting the activity of the cytokine.

4. A method according to claim 3 wherein the regulation of the cytokine is part of a more general switch from a Th2 to a Th1 cytokine profile.

5. A method according to claim 1 wherein the agent is a microorganism, or components, extract or secreted products of a microorganism.

6. A method according to claim 5 wherein the microorganism is yeast or bacteria.

7. A method according to claim 6 wherein the bacteria are probiotic bacteria.

8. A method according to claim 7 wherein the probiotic bacteria are selected from the following: Lactobacillus spp, Mycobacterium spp and Bifidbacterium.

9. A method according to claim 8 wherein the probiotic bacteria are Lactobacillus acidophilus or Mycobacterium vaccae.

10. A method according to claim 5 wherein the microorganism is alive.

11. A method according to claim 1 wherein the agent is an antibody or binding fragment thereof.

12. A method according to claim 11 wherein the antibody or binding fragment thereof is an anti-CD40 antibody or a binding fragment thereof.

13. A method according to claim 13 further comprising administration of one or more pharmaceutically active agents used to treat underlying conditions that may exacerbate the cardiovascular disorder.

14. A method according to claim 13 wherein the pharmaceutically active agent is selected from the following: a lipid-lowering drug, an anti-hypertensive agent and an anti-diabetic agent.

15. A method according to claim 13 wherein the agent used to regulate the cytokine level or activity is administered prior to, simultaneously with or subsequent to the one or more such pharmaceutically active agents.

16. A method according to claim 1 wherein the cardiovascular disorder or associated disorder is exacerbated by bacterial infection, bacterial antigens, polyclonal activators, super antigens or autoantigens.

17. A method according to claim 16 wherein the cardiovascular disorder or associated disorder is exacerbated by bacterial antigen from Chlamydia pneumoniae, Helicobacter pylori or non-typable Haemophilus influenzae.

18. A method of diagnosing, or evaluating susceptibility of a subject to, a cardiovascular disorder or associated disorder comprising detecting the level and/or activity of a cytokine associated with the disorder in the subject.

19. A method according to claim 18 wherein the level and/or activity of the cytokine is detected by analysis of circulating T-cells.

20. A method according to claim 19 wherein the cytokine is selected from the following: interferon-γ (INF-γ), interleukin-4 (IL-4), interleukin-10 (IL-10), interleukin-12 (IL-12), interleukin-13 (IL-13) and TGF-β.

21. A method of diagnosing a cardiovascular disorder or associated disorder or evaluating whether a subject is susceptible to the disorder, comprising:
(a) measuring one or more immunoglobulin levels affected by the disorder to obtain test data; and
(b) comparing the test data with reference data to evaluate whether the subject is susceptible to, or has, the cardiovascular disorder or associated disorder.

22. A method according to claim 21 wherein the immunoglobulin is IgG.

23. A method according to claim 22 wherein the IgG is IgG2 subclass.

24. A method according to claim 23 wherein the IgG2 subclass is specific for pathogenic bacteria.

25. A method according to claim 24 wherein the pathogenic bacteria are Chlamydia pneumoniae, Helicobacter pylori or non-typable Haemophilus influenzae.

26. A method according to claim 24 wherein a ratio of total IgG2 to IgG2 subclass specific antibody, or an altered ratio of total IgG2 subclass immunoglobulin to IgG2 subclass specific antibody is an indicator of the presence of or susceptibility to the cardiovascular disorder or associated disorder.

27. A method according to claim 1 wherein the cardiovascular disorder is atheroma, degenerative vascular disease or any cardiovascular condition or disease associated with inflammation of the coronary arteries including 1 to 3 coronary artery disease.

28. A method according to claim 27 wherein the atheroma is atheroma as determined by angiography, with minimal or extensive coronary atherosclerosis but stable clinical disease.

29. A method according to claim 27 wherein the atheroma is part of an unstable clinical disease associated with recent myocardial infarction or unstable angina

30. A method according to claim 1 wherein the cardiovascular associated disorder is hypertension or increased cholesterol levels.

31. Use of an agent for the manufacture of a medicament for the treatment of a cardiovascular disorder or associated disorder wherein said agent is capable of regulating the level or activity of a cytokine associated with the disorder,

32. Use according to claim 31 wherein the cytokine is selected from the group consisting of: interferon-γ (INF-γ), interleukin-4 (IL-4), interleukin-10 (IL-10), interleukin-12 (IL-12), interleukin-13 (IL-13) and TGF-β.

33. Use according to claim 31 wherein regulation of the cytokine is achieved by increasing or decreasing the level of the cytokine and/or by potentiating or inhibiting the activity of the cytokine.

34. Use according to claim 33 wherein the regulation of the cytokine is part of a more general switch from a Th2 to a Th1 cytokine profile.

35. Use according to claim 31 wherein the agent is a microorganism, or components, extract or secreted products of a microorganism.

36. Use according to claim 35 wherein the microorganism is yeast or bacteria.

37. Use according to claim 36 wherein the bacteria are probiotic bacteria.

38. Use according to claim 37 wherein the probiotic bacteria are selected from the following: Lactobacillus spp, Mycobacterium spp and Bifidobacterium.

39. Use according to claim 38 wherein the probiotic bacteria are Lactobacillus acidophilus or Mycobacterium vaccae.

40. Use according to claim 35 wherein the microorganism is alive.

41. Use according to claim 31 wherein the agent is an antibody or binding fragment thereof.

42. Use according to claim 31 wherein the cardiovascular disorder or associated disorder is exacerbated by bacterial infection, bacterial antigens, polyclonal activators, super antigens or autoantigens.

43. Use according to claim 42 wherein the cardiovascular disorder or associated disorder is exacerbated by bacterial antigen from Chlamydia pneumoniae, Helicobacterium or non-typable Haemophilus infuenzae.

44. A kit for use in a method according to claim 18

45. A method according to claim 18 wherein the cardiovascular disorder is atheroma, degenerative vascular disease or any cardiovascular condition or disease associated with inflammation of the coronary arteries including 1 to 3 coronary artery disease.

46. A method according to claim 21 wherein the cardiovascular disorder is atheroma, degenerative vascular disease or any cardiovascular condition or disease associated with inflammation of the coronary arteries including 1 to 3 coronary artery disease.

47. A method according to claim 18 wherein the cardiovascular associated disorder is hypertension or increased cholesterol levels.

48. A method according to claim 21 wherein the cardiovascular associated disorder is hypertension or increased cholesterol levels.

49. A kit for use in a method according to claim 21.
